(19) United States
(12) Patent Application Publication
(10) Pub. No.: US 2019/0167799 A1
(43) Pub. Date: Jun. 6, 2019

(54) ETHER TYPE GLYCEROPHOSPHOLIPID-CONTAINING COMPOSITION AND METHOD FOR PRODUCING THE SAME

(71) Applicant: B&S CORPORATION:KK, Tokyo (JP)

(72) Inventors: Yasuhiro SASUGA, Tokyo (JP); Masaru FUKUI, Tokyo (JP); Takehiko FUJINO, Kasuya-gun Fukuoka (JP); Shiro MAWATARI, Kasuya-gun Fukuoka (JP)

(73) Assignee: B&S CORPORATION:KK, Tokyo (JP)

(21) Appl. No.: 16/092,696

(22) PCT Filed: Apr. 27, 2016
(86) PCT No.: PCT/JP2016/063133

§ 371 Date: Oct. 10, 2018

(51) Int. Cl. A61K 47/40; A23J 7/00; A23L 33/10; A61K 31/685

(52) U.S. Cl. CPC A61K 47/40; A23J 7/00; A23V 2002/00; A61K 31/685; A23L 33/10

(57) ABSTRACT

To provide an ether-type glycerophospholipid-containing composition that has excellent heat stability and also has oxidation stability and storage stability. An ether-type glycerophospholipid-containing composition that comprises not more than 20 mass % of an ether-type glycerophospholipid and not less than 80 mass % of a cyclodextrin. Owing to this constitution, the ether-type glycerophospholipid is clathrated in the cyclodextrin. Thus, the ether-type glycerophospholipid-containing composition has highly excellent heat stability as well as temporal stability such as oxidation stability and storage stability even in a powdery form.

### FIELD OF THE INVENTION

The present invention relates to an ether type glycerophospholipid-containing composition and a method for producing the composition.

More specifically, the present invention relates to an ether type glycerophospholipid-containing composition which is excellent in thermal stability and also has oxidation stability and storage stability, and a method for producing the composition.

### BACKGROUND ART

It is known that glycerophospholipids are important as structural components of a biomembrane.

Such glycerophospholipids can be divided into subclasses of diacyl type glycerophospholipid, alkenylacyl type glycerophospholipid (plasmalogen), and alkyl ether type glycerophospholipid.

Among the glycerophospholipids, alkenylacyl type glycerophospholipid (plasmalogen) and alkyl ether type phospholipid are collectively called as ether type glycerophospholipid since they have an ether bond.

In particular, the plasmalogen having a vinyl ether bond at the 1-position of a fatty acid, which is a phospholipid distinctively contained in large amounts in cranial nerve cells and myocardia, is a lipid component that has been attracting attention in recent years.

It has been reported that such plasmalogen has the vinyl ether structure, which has a distinctive biological function, in the molecule, and exhibits antioxidative properties by capturing active oxygen, radicals or metal ions, and, in addition, is involved in the fluidity and flexibility of cell membranes (in particular, synaptic membranes of nerve cells) (Non-patent Document 1).

It has also been reported that brains with Alzheimer's disease have a significantly lower plasmalogen type phospholipid concentration than brains of healthy adults, by as much as approximately 30% (Non-patent Documents 1 and 2).

Under such circumstances, it has been proposed that foods and beverages or pharmaceuticals are produced so as to contain plasmalogen or ether type glycerophospholipid in order to improve or prevent diseases, such as Alzheimer's disease (Patent Documents 1 to 4).

The highly purified ether type glycerophospholipid is clayey and is basically very difficult to handle and is easily degraded because the vinyl ether bond within the structure or the polyunsaturated fatty acid included therein is considerably oxidized.

On the other hand, it has been proposed that a high-fluidity powder composition having a high fluidity, slight fishy smell, and high stability, which contains diglyceride-3-phosphate derived from fish and shellfish and derivatives thereof, in Japanese Patent Kokai Application Publication No. JP2007-161834A (Patent Document 5).

The high-fluidity powder composition contains 20 to 90 mass % of diglyceride-3-phosphate derived from fish and shellfish and derivatives thereof, and 10 to 80 mass % of a starch decomposition product containing 1 to 50 mass % of a and/or γ-cyclodextrin, wherein the amount of volatile components at a temperature of 25° C. is 1.0 ppm or less.

### PRIOR ART DOCUMENTS

### Patent Documents

Patent Document 1: Japanese Patent Kokai Application Publication No. JP2003-003190A
Patent Document 2: Japanese Patent Kokai Application Publication No. JP2003-012520A
Patent Document 3: Japanese Patent Kokai Application Publication No. JP2004-026803A
Patent Document 4: Japanese Patent Kokai Application Publication No. JP2013-053109A
Patent Document 5: Japanese Patent Kokai Application Publication No. JP2007-161834A

### Non-Patent Documents

Non-patent Document 1: MIYAZAWA Taiki et. al, “A challenge for preventing senile dementia with marine plasmalogen”, FOOD STYLE 21, 14(4): p 29-31(2010)
Non-patent Document 2: Braverman N E and Moser A B, Functions of plasmalogen lipids in health and disease, BBA, 1822: p 1442-1452(2012)

### SUMMARY OF THE INVENTION

### Problem to be Solved by the Invention

There is a problem in that degradation of the ether type glycerophospholipid, especially in a purified state, is accelerated at the ordinary temperature or more, and therefore the ether type glycerophospholipid is very difficult to store in a stable state and needs to be frozen or refrigerated on the storage thereof.

Such a problem is particularly significant in the case of ethanolamine type ether glycerophospholipid.

The Patent Document 5 exemplifies several compounds as diglyceride-3-phosphate. However, in Examples thereof, the only phospholipid that the effect has been confirmed specifically is a phospholipid derived from scallop, which may contain many varieties of phospholipids, and no effects of ether type glycerophospholipid have been confirmed at all.

In addition, the Patent Document 5 has a structure different from the present invention in that the cyclodextrin used in the Patent Document 5 is essentially in the form of a mixture containing a starch decomposition product.

In addition, the Patent Document 5 discloses the effect of suppressing an increase in the acid value; however, it does not disclose or suggest improvement of stability, in particular, thermal and oxidation stability.

The amount of cyclodextrin added relative to phospholipid is different from that of the present invention, and hence an object of improving stability, particularly thermal and oxidation stability, remains unachieved.

Therefore, it is required to provide an ether type glycerophospholipid which is excellent in an improving/preventive effect to diseases such as Alzheimer's disease, in which degradation is suppressed or prevented, and which is improved in thermal stability, oxidation stability, and storage stability.

Under such circumstances, the present inventors have studied for an objective to provide a method of stabilizing ether type glycerophospholipid.

As a consequence, the present inventors found that, by including ether type glycerophospholipid in cyclodextrin, ether type glycerophospholipid having, even in powder form, high thermal stability, high oxidation stability, and high storage stability can be produced. Thus, the present invention has been completed.

### Solution to the Problem

The present invention according to claim 1 is characterized in,

an ether type glycerophospholipid-containing composition containing:

20 mass % or less of ether type glycerophospholipid; and

80 mass % or more of cyclodextrin.

The present invention according to claim 2 is characterized in,

the ether type glycerophospholipid-containing composition according to claim 1,

wherein the ether type glycerophospholipid is included in the cyclodextrin.

The present invention according to claim 3 is characterized in,

the ether type glycerophospholipid-containing composition according to claim 1 or 2,

wherein the ether type glycerophospholipid-containing composition is in powder form.

The present invention according to claim 4 is characterized in,

the ether type glycerophospholipid-containing composition according to any one of claims 1 to 3,

wherein the cyclodextrin is γ-cyclodextrin.

The present invention according to claim 5 is characterized in,

the ether type glycerophospholipid-containing composition according to any one of claims 1 to 4,

wherein the ether type glycerophospholipid-containing composition has thermal and oxidation stability with which a degradation ratio of less than 50% can be maintained while being treated at a temperature of 60° C. for 96 hours.

The present invention according to claim 6 is characterized in,

the ether type glycerophospholipid-containing composition according to claim 1, containing:

less than 20 mass % of ether type glycerophospholipid; and

more than 80 mass % of cyclodextrin.

The present invention according to claim 7 is characterized in,

a method for producing an ether type glycerophospholipid-containing composition containing

mixing together 20 mass % or less of ether type glycerophospholipid and 80 mass % or more of cyclodextrin in the presence of water and/or ethanol.

The present invention according to claim 8 is characterized in,

the method for producing an ether type glycerophospholipid-containing composition according to claim 7,

wherein a mixture obtained is subjected to drying.

The present invention according to claim 9 is characterized in,

the method for producing an ether type glycerophospholipid-containing composition according to claim 8

wherein the drying is performed by freeze drying or spray drying.

The present invention according to claim 10 is characterized in,

an ether type glycerophospholipid-stabilizing agent,

wherein 80 mass % or more of the cyclodextrin is incorporated into 20 mass % or less of ether type glycerophospholipid.

### Advantageous Effects of the Invention

An ether type glycerophospholipid-containing composition according to the present invention contains 20 mass % or less of ether type glycerophospholipid, and 80 mass % or more of cyclodextrin.

Thus, the ether type glycerophospholipid-containing composition, even in powder form, has extremely superior stability with time such as thermal stability, oxidation stability and storage stability since the ether type glycerophospholipid is included in the cyclodextrin.

In the composition described above, when γ-cyclodextrin is selected as the cyclodextrin, the ether type glycerophospholipid can exhibit its function more effectively since the γ-cyclodextrin has properties of being water-soluble and absorbable through the small intestine.

In particular, the composition described above, even in powder form, has thermal and oxidation stability with which a degradation ratio of less than 50% is maintained while being treated at a temperature of 60° C. for 96 hours, and hence also has high storage stability.

The ether type glycerophospholipid-containing composition described above can be easily produced by, in particular, mixing together 20 mass % or less of ether type glycerophospholipid and 80 mass % or more of cyclodextrin in the presence of water and/or ethanol.

In addition, the mixture obtained above may be subjected to a drying step such as freeze drying or spray drying, so that an ether type glycerophospholipid-containing composition in powder form can be easily obtained.

An ether type glycerophospholipid-stabilizing agent according to the present invention contains cyclodextrin as an active ingredient, and is the one that 80 mass % or more of the cyclodextrin is incorporated into 20 mass % or less of the ether type glycerophospholipid.

Thus, this stabilizing agent enables suppression or prevention of degradation of ether type glycerophospholipid, especially in powder form, to thereby achieve long-term storage.

### BRIEF DESCRIPTION OF THE DRAWINGS

FIG. 1 shows an HPLC chart depicting a purified extract of ether type glycerophospholipid derived from scallop.

FIG. 2 shows an HPLC chart depicting ether type glycerophospholipid derived from scallop which is contained in an ether type glycerophospholipid-containing composition obtained in Example 1.

FIG. 3 shows an HPLC chart depicting ether type glycerophospholipid derived from scallop which is contained in an ether type glycerophospholipid-containing composition obtained in Example 2.

FIG. 4 shows an HPLC chart depicting ether type glycerophospholipid derived from scallop which is contained in an ether type glycerophospholipid-containing composition obtained in Example 3.

FIG. 5 shows an HPLC chart depicting ether type glycerophospholipid derived from scallop which is contained in an ether type glycerophospholipid-containing composition obtained in Example 4.

FIG. 6 shows an HPLC chart depicting ether type glycerophospholipid derived from scallop which is contained in an ether type glycerophospholipid-containing composition obtained in Comparative example 2.

FIG. 7 shows the stability test results of ether type glycerophospholipid derived from scallop which is contained in an ether type glycerophospholipid-containing composition obtained in Example 1.

FIG. 8 shows the stability test results of ether type glycerophospholipid derived from scallop which is contained in an ether type glycerophospholipid-containing composition obtained in Example 2.

FIG. 9 shows the stability test results of ether type glycerophospholipid derived from scallop which is contained in an ether type glycerophospholipid-containing composition obtained in Examples 3 and 4.

FIG. 10 shows the stability test results of ether type glycerophospholipid derived from scallop which is contained in an ether type glycerophospholipid-containing composition obtained in Comparative example 2.

### DESCRIPTION OF EMBODIMENTS

An ether type glycerophospholipid-containing composition according to an embodiment of the present invention will be described hereinbelow.

Although the present invention is described mainly by preferred representative examples, the present invention is not limited to such examples.

Further, in describing the present invention, the following abbreviations may be employed.

PE: phosphatidyl ethanolamine (a kind of diacyl type glycerophospholipids)

PC: phosphatidyl choline (a kind of diacyl type glycerophospholipids)

CAEP: ceramideaminoethylphosphonic acid

pls: plasmalogen

plsPE: ethanolamine plasmalogen

plsPC: choline plasmalogen

PLA1: phospholipase A1

Chol: cholesterol

This ether type glycerophospholipid-containing composition according to the present invention contains 20 mass % or less of ether type glycerophospholipid and 80 mass % or more of cyclodextrin.

The composition having such a structure thus has extremely superior stability with time such as thermal stability, oxidation stability and storage stability.

The ether type glycerophospholipid in the composition according to the present invention exerts an effect of improving or preventing diseases such as Alzheimer's disease.

The ether type glycerophospholipid may be selected from various ether type glycerophospholipids.

In the present invention, in particular, a glycerophospholipid that has a vinyl ether bond (alkenyl bond) or an ether bond (alkyl bond) at the 1-position of the glycerol backbone (sn-1) is selected.

General formulas of ether type glycerophospholipids are described below.

A compound represented by Formula (1) is an alkenylphospholipid (plasmalogen), while a compound represented by Formula (2) is an alkylphospholipid.

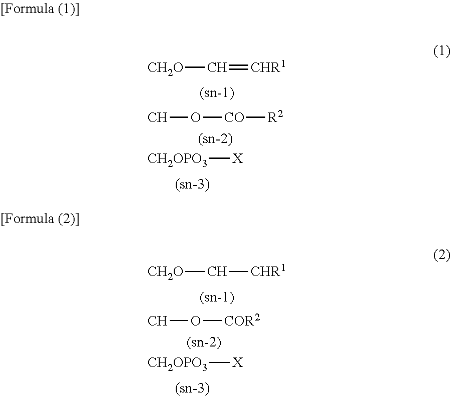

In the above formulas, R¹ represents an aliphatic hydrocarbon group.

R¹ is usually an aliphatic hydrocarbon group having 14 to 18 carbon numbers.

R² represents an aliphatic hydrocarbon group, which may be bound to polyunsaturated fatty acid, such as arachidonic acid (ARA), docosahexaenoic acid (DHA), eicosapentaenoic acid (EPA), and the like.

Further, X represents a nitrogen-containing alcohol group or a polyol group in the above formulas.

Examples of the nitrogen-containing alcohol group include hydrogen, a serine group, an ethanolamine group, an N-methylethanolamine group, a dimethylethanolamine group, and a trimethylethanolamine group.

Examples of the polyol group include a glycerol group, a glycerophosphate group, a glycerophosphate phosphatidyl group, an inositol group, an inositol phosphate group, and an inositol diphosphate group.

The ether type glycerophospholipid may be a natural substance extracted/purified from a biological material (organism), or may be a chemical synthetic product.

The biological material may be anything and not limited in particular, as long as it contains the ether type glycerophospholipid.

Example may include animals, plants and microorganisms.

The biological material is preferably selected from animals and their tissues since they have a higher ether type glycerophospholipid content than those of plant tissues and microorganisms and are easily available in large quantities at a low cost.

The exemplary animals may be mammals, birds, fish and shellfish, etc.

It is preferred that the mammals are domestic animals due to supply stability and safety.

Examples include the mammals such as cows, pigs, horses, goats, sheep, dears, camels, lamas, and the like, and domestic poultry such as chicken, ducks, turkeys, ostriches, and the like.

With regard to mammals, primary tissues containing ether type glycerophospholipid may include skins, brains, intestines, hearts, genitals, etc.

The fish and shellfish are preferably those farmable, in other words, culturable. Examples include:

1) fishes such as yellowtail, red sea bream, silver salmon (Oncorhynchus kisutch), amberjack, flatfish, Sphoeroides rubripes, striped jack, Japanese horse mackerel, gold-striped amberjack, Lateolabrax maculatus, sea bass, Rachycentron canadum, bluefin tuna, Japanese tiger prawns, carp, eel, rainbow trout, sweetfish (Plecoglossus altivelis), Oncorhynchus masou, Oncorhynchus rhodurus, Salvelinus leucomaenis pluvius, Salvelinus leucomaenis leucomaenis, and Salvelinus leucomaenis japonicus Oshima, etc.;

2) crustaceans such as Japanese tiger prawns, giant tiger prawns, Chinese white shrimp (Fenneropenaeus chinensis), and swimming crab (Portunus trituberculatus), etc.; and

3) shellfishes such as abalones, horned turbans, scallops, and oysters, etc.

Of these, more preferred examples are shellfish such as abalones, turban shells, scallops, and oysters.

In particular, scallops are preferably selected because they have low neutral lipid content in the whole lipids, have high phospholipid content, and also have high ether type glycerophospholipid content in the phospholipids.

With regard to the fish and shellfish, primary tissues containing ether type glycerophospholipid may include internal organs, gonad, and muscles, etc.

Examples of the microorganisms include bacteria of Propionibacterium.

With regard to bacteria, “tissue” is bacteria by itself.

The method for producing the ether type glycerophospholipid is not particularly limited, and is appropriately selected from the viewpoint of ease of production and costs, for example.

For example, the ether type glycerophospholipid can be produced by well-known methods such as the method disclosed in Japanese Unexamined Patent Application Publication No. 2010-065167.

The cyclodextrin has a property of taking a specific molecule within its void, and includes the ether type glycerophospholipid to form a clathrate.

Examples of the cyclodextrin include α-cyclodextrin, β-cyclodextrin, and γ-cyclodextrin. These may be used alone or in combination of two or more thereof.

The γ-cyclodextrin is preferred because it has properties of being water-soluble and absorbable through the small intestine, and easily demonstrates the function of the ether type glycerophospholipid effectively.

Incidentally, such cyclodextrins may be chemically modified (for example, methylation or acetylation).

Such cyclodextrins can be purchased from CycloChem Co., Ltd., for example.

In the present invention, the composition contains 20 mass % or less of the ether type glycerophospholipid, and 80 mass % or more of the cyclodextrin.

The proportions of the ether type glycerophospholipid and the cyclodextrin at least satisfy the above-described ranges.

An optimum composition ratio can be selected from the viewpoint of, for example, the stability and various properties of the resultant composition, or the cyclodextrin content.

The composition preferably contains less than 20 mass % of the ether type glycerophospholipid, and more than 80 mass % of the cyclodextrin, more preferably 1 to 15 mass % of the ether type glycerophospholipid, and 99 to 85 mass % of the cyclodextrin, still more preferably 5 to 10 mass % of the ether type glycerophospholipid, and 95 to 90 mass % of the cyclodextrin.

Incidentally, in view of the effective intake per day, the ether type glycerophospholipid is preferably contained at 0.05 mass % as a lower limit.

The ether type glycerophospholipid-containing composition having such a structure can be produced by various production methods.

For example, the composition can be obtained, by a well-known method such as the emulsification method, the saturated aqueous solution method, the kneading method, or the mixing-pulverization method, such that the specific amount of ether type glycerophospholipid and the specific amount of cyclodextrin are mixed together to include the ether type glycerophospholipid in the cyclodextrin.

Specifically, the specific amount of ether type glycerophospholipid and the specific amount of cyclodextrin can be mixed together in the presence of water and/or ethanol.

When the specific amount of ether type glycerophospholipid and the specific amount of cyclodextrin are mixed together, an antioxidant such as vitamin E may be added.

When the antioxidant is added, oxidation of ether type glycerophospholipid during production can be suppressed. As a result, the amount of ether type glycerophospholipid contained in the composition increases.

Incidentally, the amount of the antioxidant added relative to the amount of ether type glycerophospholipid contained is preferably 0.010 to 0.5 mass %.

Examples of the vitamin E include tocopherol and tocotrienol.

These may be in the form of α, β, γ, or δ.

In the present invention, after the ether type glycerophospholipid and the cyclodextrin are mixed together to form a clathrate of the ether type glycerophospholipid and the cyclodextrin, the water content can be removed from the clathrate by a well-known drying method such as the freeze drying method or the spray drying method to obtain powder.

The form of the ether type glycerophospholipid-containing composition according to the present invention is not particularly limited.

The form can be selected from various forms such as powder, granules, liquid, latex, cream, and gel.

Of these, forms of granules or powder are preferred from the viewpoint of ease of handling; however, the form is not particularly limited.

Incidentally, in the ether type glycerophospholipid-containing composition according to the present invention, degradation of the ether type glycerophospholipid in this composition is suppressed or prevented, so that the composition can also be suitably used in powder form intended for long-term storage.

Such an ether type glycerophospholipid-containing composition contains ether type glycerophospholipid as an active ingredient.

Thus, the composition is significantly effective for treating or improving cranial nerve diseases such as Alzheimer's disease, Parkinson's disease, depression, and schizophrenia, metabolic syndromes such as diabetes, various infectious diseases, and immune disorders.

In addition, with regard to the ether type glycerophospholipid-containing composition, ether type glycerophospholipid (especially, in clayey form) is generally degraded in a degradation ratio of approximately 80% by being treated in an environment at a temperature of 60° C. for 96 hours; by contrast, even in powder form, the degradation of ether type glycerophospholipid is maintained to be less than 50% even if it is allowed to stand under an environment at a temperature of 60° C. for 96 hours.

Thus, the composition has extremely superior stability with time, in particular, thermal stability, oxidation stability, and storage stability.

The ether type glycerophospholipid-containing composition may be applied to a beverage and food product as a material and to a pharmaceutical composition as an ingredient.

Such a beverage and food product and a pharmaceutical composition may be produced in accordance with a well-known method.

Furthermore, the ether type glycerophospholipid-containing composition may be applied to various forms of beverage and food products whether well-known or that would be developed in the future, as deemed fit.

Similarly, the composition may be applied to a form of functional foods or that of specified health uses.

The exemplary form of beverage and food products includes:

1) beverages such as soft drinks, green tea beverages, black tea beverages, coffee beverages, fermented tea beverages (such as oolong tea), vegetable juices, cow's milk, milk-based drinks, fermented milk beverages, health drinks, sports drinks, jelly drinks, and alcohol beverages;

2) common foods such as jelly foods, frozen desserts, cakes, candies, caramels, chewing gum, Japanese-style confectionery, snack confectionery, chocolates, soda pop flavored sweets, gummi candies, puddings, yogurt, soups, miso soup, rice, rice balls, processed meat, bread, udon, buckwheat noodles, ramen (Chinese noodle), pasta, konjak, pickles, fermented soybeans, deep frying flour, flour, dogtooth violet starch, gelatin, bread crumbs, fish-paste products, retort foods, frozen foods, chilled foods, and instant foods;

3) seasonings such as sprinkled seasonings, sauce, soy sauce, fish sauce, miso, sake for cooking, vinegar, sweet sake for seasoning, oyster sauce, tare sauce, mayonnaise, ketchup, salt, spices, herbs, curry powder, cooking oil, sauce for noodles, taste enhancers, hot condiments, and flavor enhancers;

4) processed foods such as capsules, tablets, sugar-coated tablets, granules, powders, liquid agents, edible films, and jellies; and

other various products.

In case that the ether type glycerophospholipid-containing composition is applied to a pharmaceutical composition as an ingredient, glycerophospholipid-containing composition, which is an active ingredient, may contain pharmacologically acceptable bases, carriers or additives (such as diluents, binders, disintegrants, lubricants, solvents, sweeting agents, coloring agents, flavorings, corrigents, surfactants, humectants, preservatives, pH adjusting agents, and thickening agents), in a range that does not impair the effects of the present invention such as thermal stability, oxidation stability and the like, if necessary.

Such bases, carriers, additives, and the like are specifically described, for example, in Japanese Pharmaceutical Excipients Directory 2000 (Yakuji Nippo, Limited), and, for example, those described therein may be used.

The preparation form thereof is also not particularly limited. The active component and the other components may be mixed together by using a hitherto known manner to form into a preparation form, such as tablets, coated tablets, powders, granules, fine granules, capsules, pills, liquid agents, suspensions, emulsions, jellies, chewable tablets, or soft tablets.

Although it is easy and convenient to use the ether type glycerophospholipid-containing composition by mixing it into a product used, it is as a matter of course that a certain amount of the ether type glycerophospholipid is required to achieve the above effects.

In the ether type glycerophospholipid-containing composition, the intake amount or dosage of the ether type glycerophospholipid, which is an active ingredient, varies depending on, for example, the age, body weight, constitution, or condition of the subject, the dosage form, administration route, or intake or dosing (administration) period.

In such a case where, for example, oral administration is performed, in general, the amount thereof is preferably set within a range of 0.05 to 50 mg, and more preferably 0.1 to 10 mg per day for an adult (body weight: approximately 60 kg).

Incidentally, the intake (administration) may be performed once, or performed multiple per day separate times (preferably, two or three times).

The present invention provides a method for stabilizing ether type glycerophospholipid in which 80 mass % or more of the cyclodextrin is incorporated into 20 mass % or less of the ether type glycerophospholipid to include the ether type glycerophospholipid in the cyclodextrin.

This invention is easily worked with referring to the above description.

The present invention also provides an ether type glycerophospholipid-stabilizing agent containing cyclodextrin as an active ingredient,

wherein 80 mass % or more of the cyclodextrin is incorporated into 20 mass % or less of the ether type glycerophospholipid.

This invention is easily worked with referring to the above description.

### EXAMPLES

The followings describe the present invention with reference to Examples in details on ether type glycerophospholipid-containing compositions.

However, the present invention is not limited to these Examples.

### Production Example

(Production of Ether Type Glycerophospholipid Derived from Scallop)
(1) Extraction of Ether Type Glycerophospholipid Derived from Scallop

20 kg fresh weight of raw scallop mantles were treated in boiling water for 2 minutes to obtain approximately 5 kg of boiled scallop mantles.

The boiled scallop mantles thus obtained were chopped. Next, to the boiled scallop mantles thus chopped, 10 L of an enzyme solution (1.5% Kokulase P (registered trademark; manufactured by MITSUBISHI-CHEMICAL FOODS CORPORATION), 1.5% PLA1, 0.25 M citric acid buffer solution, pH 5.2) was added, ground by a blender, and homogenized, and subsequently subjected to enzyme treatment at a temperature of 50° C. for 2 hours.

To the treated solution, 35 L of hexane/2-propanol mixture (3:2) was added, and stirred for 10 minutes.

Subsequently, to the mixture thus obtained, 20 L of sodium sulfate (1 g/15 mL) was added, stirred for 5 minutes, and then placed.

Out of two separate layers, approximately 21 L of upper hexane layer was separated.

From the hexane layer thus obtained, the solvent was distilled away by a rotary evaporator to obtain approximately 100 g of crude extract as a lipid fraction.

To the crude extract thus obtained, 1.6 L of acetone was added, sufficiently stirred, and subsequently placed at a temperature of −30° C. for 1 hour or longer in a refrigerator.

Subsequently, the precipitation was collected by decantation and filtration processes, and acetone was completely distilled away under a reduced pressure, to obtain 58 g of purified ether type glycerophospholipid derived from scallop (purified extract).

(2) Analysis of Ether Type Glycerophospholipid Derived from Scallop by HPLC

HPLC analysis was provided on the solution, in which 2 mg of the purified extract obtained above was dissolved in 1 mL of hexane/2-propanol mixture (3:2), under the following conditions.

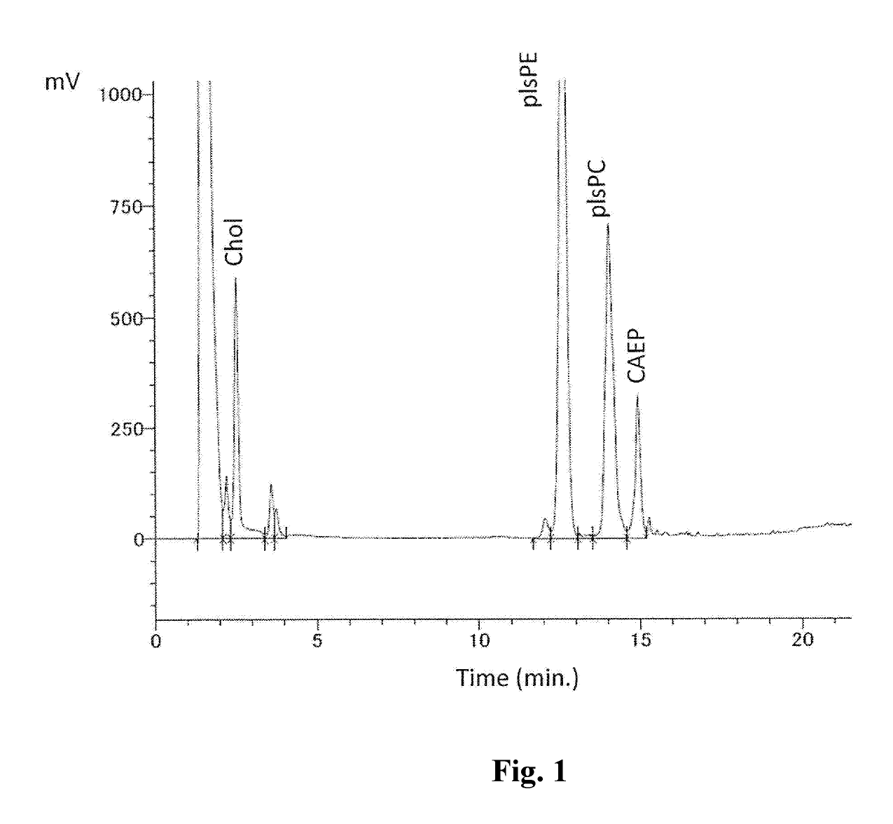

The results are shown in FIG. 1.

### <Conditions for HPLC>

1) Device used: Shimadzu LC-10ADvp (manufactured by SHIMADZU CORPORATION)

2) Column: LiChrospher Diol 100 (5 μm, 250-4, manufactured by Merck Millipore)

3) Flow rate: 1.0 mL/min

4) Column temperature: temperature of 50° C.

5) Detector: ELSD-LTII (Evaporative Light Scattering Detector) (manufactured by SHIMADZU CORPORATION)

6) Drift tube temperature: temperature of 50° C.

7) Mobile phase:
(A) hexane/2-propanol/acetic acid (82:17:1, v/v)+0.08% trimethylamine)
(B) 2-propanol/water/acetic acid (85:14:1)+0.08% triethylamine)

8) Gradient: (B) 5%, 0 min→(B) 65%, 20 min→(B) 85%, 21 min→(B) 85%, 22 min→(B) 5%, 25 min

### <Results>

According to FIG. 1, it is found that the lipid fraction obtained in the Production example hardly contain diacyl type glycerophospholipid, and 75% or more of the lipid fraction is ether type glycerophospholipid, which is highly-purified ether type glycerophospholipid.

### Example 1

(Production of Ether Type Glycerophospholipid Composition Derived from Scallop)

To 1.0 g of the highly-purified ether type glycerophospholipid derived from scallop, which was obtained in the Production example, 90 mL of water and 10 mL of ethanol were added, and subjected to a sufficiently ultrasonic homogenizer treatment to obtain a homogeneous emulsion.

To the emulsion thus obtained, 9 g of γ-cyclodextrin (CAVMAX W8 Food, manufactured by CycloChem Co., Ltd.) was added, and stirred at room temperature for 1 hour.

The composition thus obtained was frozen at a temperature of −30° C., dried with a freeze dryer for 48 hours, and ground by a domestic mixer (Millser 800DG, manufactured by Iwatani Corporation) to obtain an ether type glycerophospholipid composition derived from scallop in powder form.

### Comparative Example 1

The ether type glycerophospholipid derived from scallop, which was obtained in the Production example, was defined as Comparative example 1.

### Example 2

(Production of Ether Type Glycerophospholipid Composition Derived from Scallop)

To 8.0 g of the highly-purified ether type glycerophospholipid derived from scallop, which was obtained in the Production example, 360 mL of water and 40 mL of ethanol were added, and subjected to a sufficiently ultrasonic homogenizer treatment to obtain a homogeneous emulsion.

To the emulsion thus obtained, 32 g of γ-cyclodextrin (CAVMAX W8 Food, manufactured by CycloChem Co., Ltd.) was added, and stirred at room temperature for 1 hour.

The composition thus obtained was frozen at a temperature of −30° C., dried with a freeze dryer for 48 hours, and ground by a domestic mixer (Millser 800DG, manufactured by Iwatani Corporation) to obtain an ether type glycerophospholipid composition derived from scallop in powder form.

### Example 3

(Production of Ether Type Glycerophospholipid Composition Derived from Scallop)

To 25.9 g of the highly-purified ether type glycerophospholipid derived from scallop, which was obtained in the Production example, 1,235 mL of water and 65 mL of ethanol were added, and subjected to a sufficiently ultrasonic homogenizer treatment to obtain a homogeneous emulsion.

To the emulsion thus obtained, 259.0 g of γ-cyclodextrin (CAVMAX W8 Food, manufactured by CycloChem Co., Ltd.) was added, and stirred at room temperature for 1 hour.

The composition thus obtained was frozen at a temperature of −30° C., dried with a freeze dryer for 48 hours, and ground by a domestic mixer (Millser 800DG, manufactured by Iwatani Corporation) to obtain an ether type glycerophospholipid composition derived from scallop in powder form.

### Example 4

(Production of Ether Type Glycerophospholipid Composition Derived from Scallop)

To 25.9 g of the highly-purified ether type glycerophospholipid derived from scallop, which was obtained in the Production example, vitamin E oil and fat having a mass ratio of 0.5% (RIKEN E-Oil Super 60, manufactured by RIKEN VITAMIN Co., Ltd.), 1,140 mL of water, and 60 mL of ethanol were added, and subjected to a sufficiently ultrasonic homogenizer treatment to obtain a homogeneous emulsion.

To the emulsion thus obtained, 233.0 g of γ-cyclodextrin (CAVMAX W8 Food, manufactured by CycloChem Co., Ltd.) was added, and stirred at room temperature for 1 hour.

The composition thus obtained was frozen at a temperature of −30° C., dried with a freeze dryer for 48 hours, and ground by a domestic mixer (Millser 800DG, manufactured by Iwatani Corporation) to obtain an ether type glycerophospholipid composition derived from scallop in powder form.

### Comparative Example 2

(Production of Ether Type Glycerophospholipid Composition Derived from Scallop)

To 12.0 g of the highly-purified ether type glycerophospholipid derived from scallop, which was obtained in the Production example, 360 mL of water and 40 mL of ethanol were added, and subjected to a sufficiently ultrasonic homogenizer treatment to obtain a homogeneous emulsion.

To the emulsion thus obtained, 36 g of γ-cyclodextrin (CAVMAX W8 Food, manufactured by CycloChem Co., Ltd.) was added, and stirred at room temperature for 1 hour.

The composition thus obtained was frozen at a temperature of −30° C., dried with a freeze dryer for 48 hours, and ground by a domestic mixer (Millser 800DG, manufactured by Iwatani Corporation) to obtain an ether type glycerophospholipid composition derived from scallop in powder form.

### Test Example 1

The ether type glycerophospholipid compositions derived from scallop in powder form, which was obtained in the Examples 1 to 4 and the Comparative example 2, were subjected to a comparison test of structure in accordance with the following test method.

### <Test Method>

To 30 mg of the ether type glycerophospholipid composition derived from scallop thus obtained in powder form, 3 mL of methanol was added, and subjected to shaking treatment for 3 hours to extract ether type glycerophospholipid derived from scallop.

HPLC analysis was provided on the extract thus obtained under the conditions similar to Example 1.

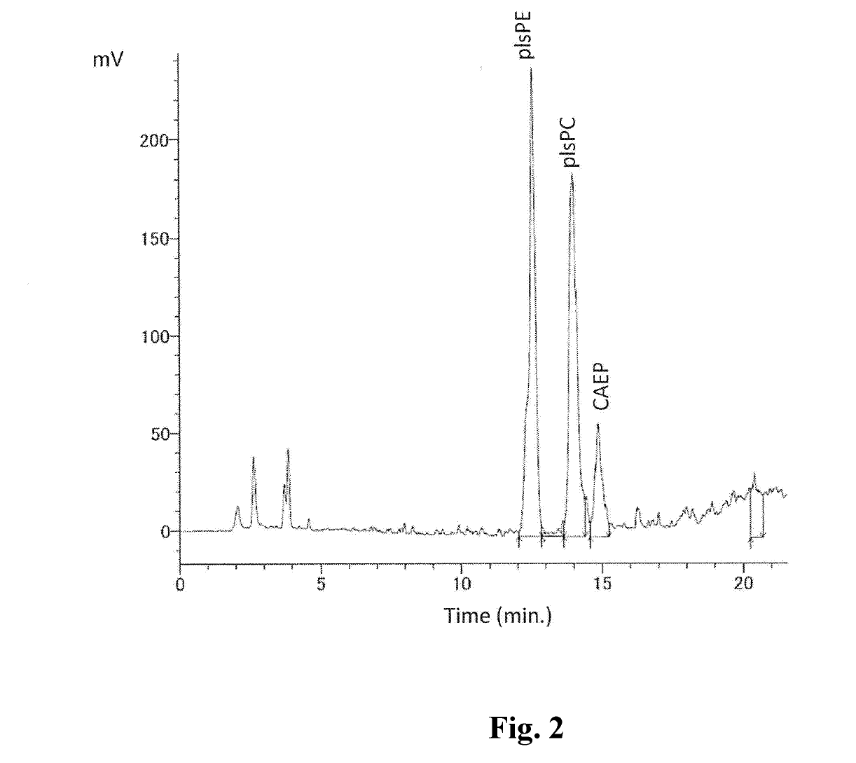

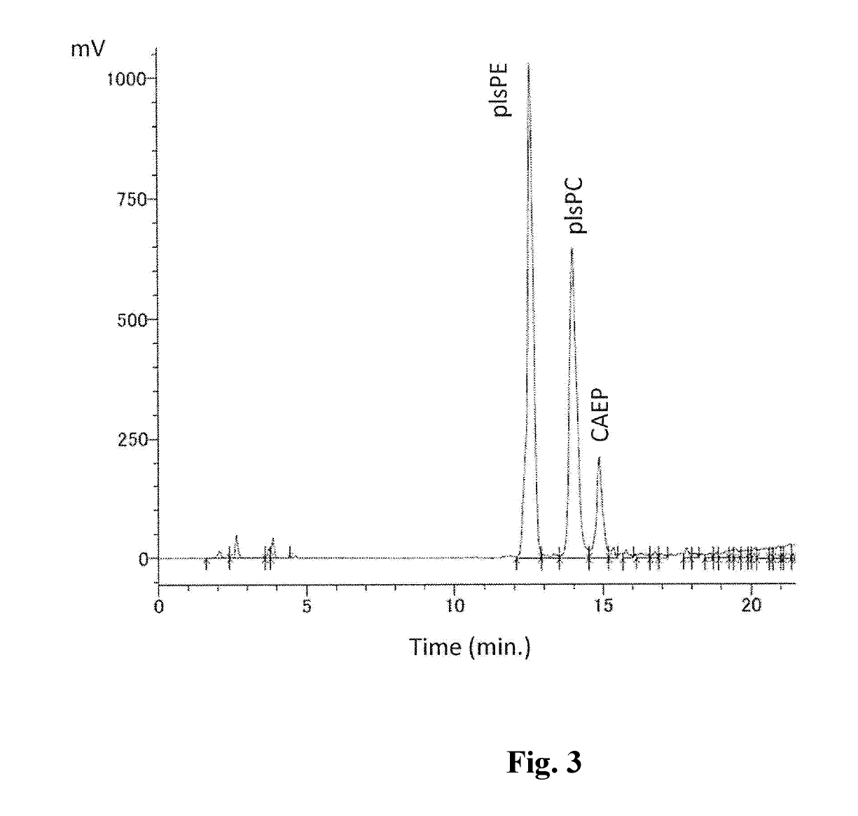

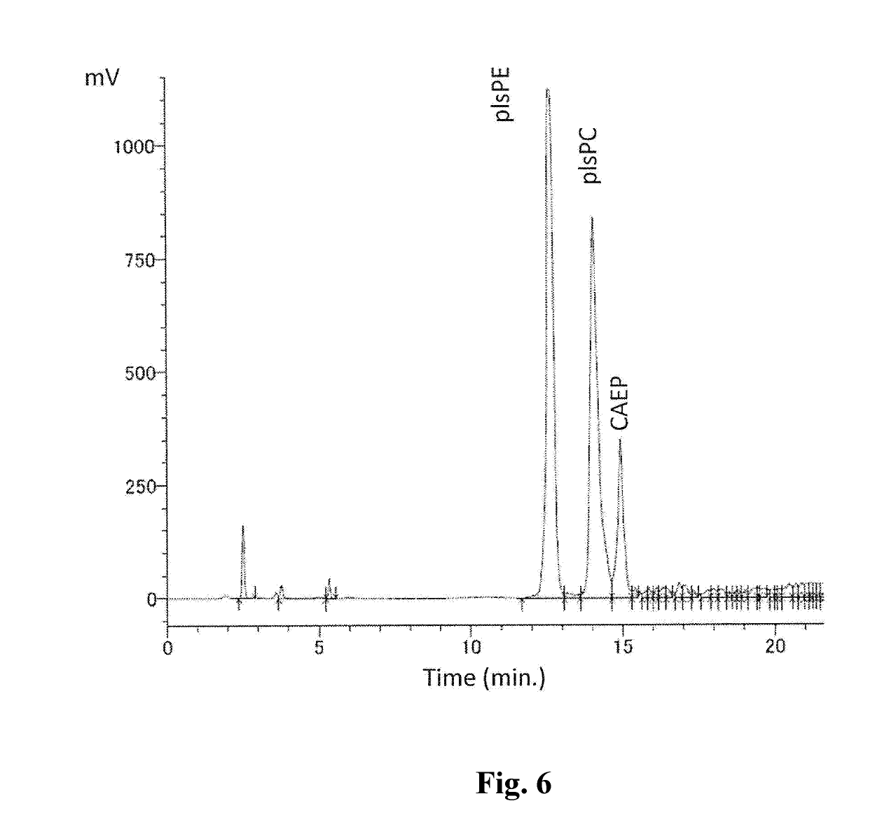

The results are shown in FIGS. 2 to 6.

### <Results>

In each of the ether type glycerophospholipid compositions derived from scallop, which were obtained in Examples 1 to 4 as well as Comparative example 2, the ether type glycerophospholipid derived from scallop is included in γ-cyclodextrin.

According to FIGS. 2 to 6, it is found that the chromatograms of ether type glycerophospholipids contained in the ether type glycerophospholipid compositions according to the present invention are the same as the chromatogram of the ether type glycerophospholipid obtained in Production example, that is, untreated ether type glycerophospholipid which is not treated with γ-cyclodextrin.

As described above, apparently, the ether type glycerophospholipid compositions according to the present invention does not cause the variations in the composition during or after the treatment with cyclodextrin, and can express the effects equal to the untreated ether type glycerophospholipid which is not treated with cyclodextrin.

### [Test Example 2] Evaluation of Effect of the Presence or Absence of Addition of Vitamin E

The ether type glycerophospholipid compositions derived from scallop in powder form, which were obtained in the Examples 3 and 4, were subjected to a test for confirming an effect of the presence or absence of addition of vitamin E in accordance with the following Test method.

### <Test Method>

To 30 mg of the ether type glycerophospholipid composition derived from scallop thus obtained in powder form, 3 mL of methanol was added, and subjected to shaking treatment for 3 hours to extract ether type glycerophospholipid derived from scallop.

HPLC analysis was provided on the extract thus obtained under the conditions similar to Example 1.

### <Results>

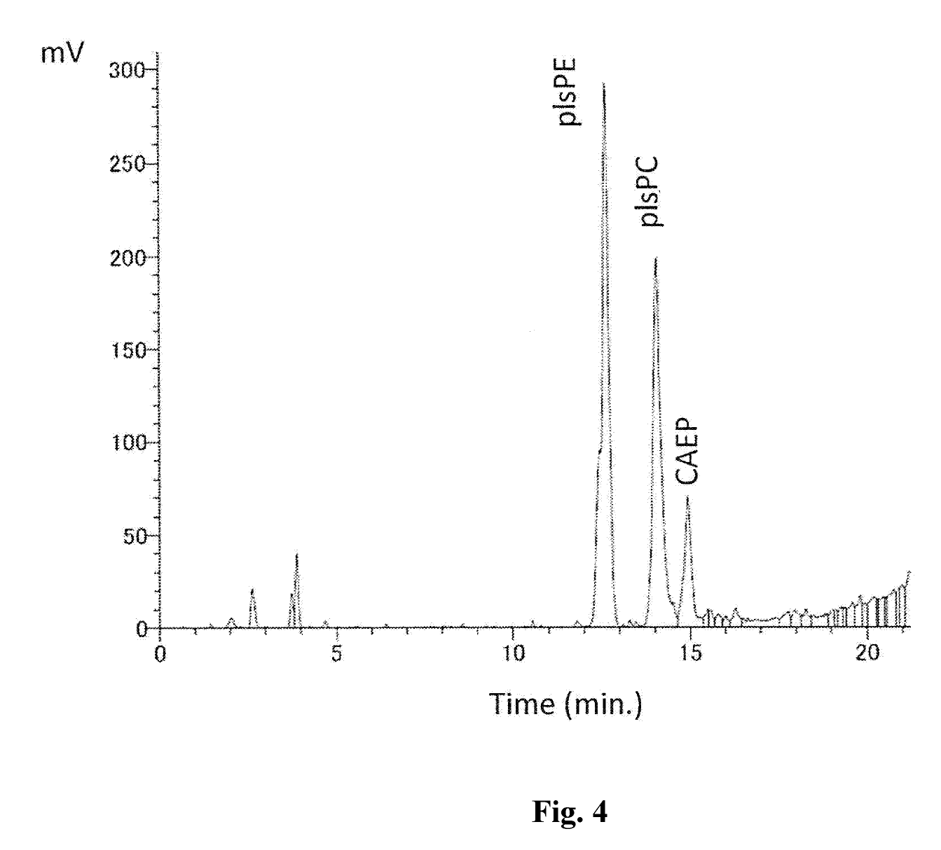

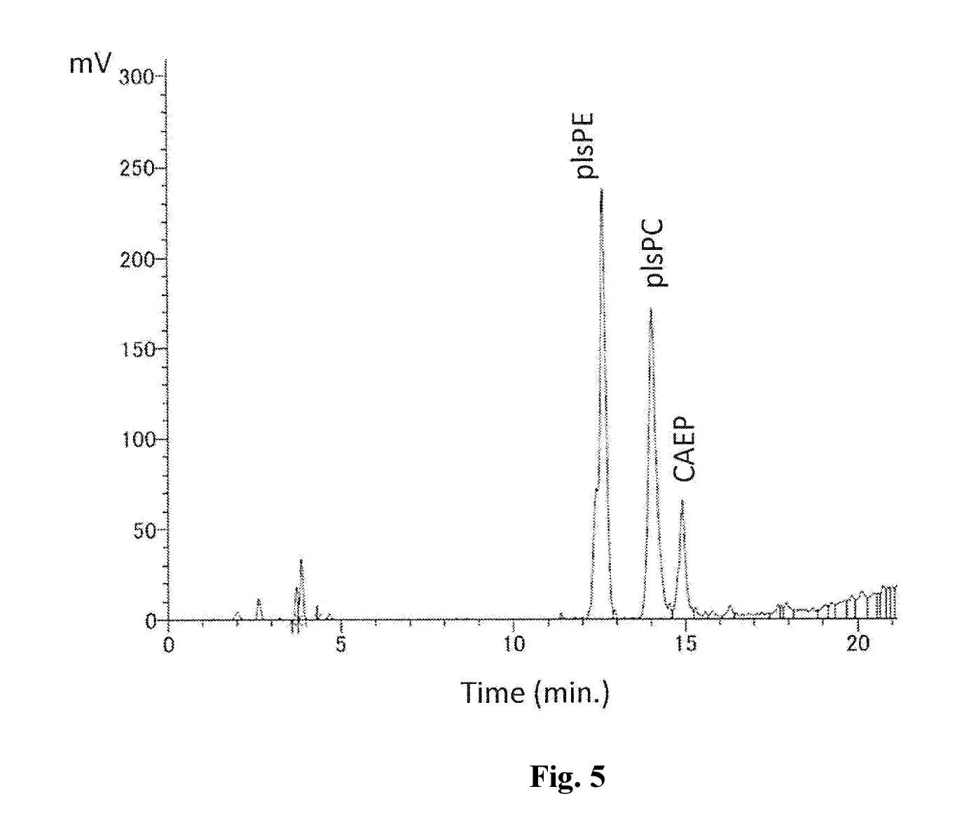

According to FIGS. 4 and 5, the amount of ether type glycerophospholipid contained in the ether type glycerophospholipid composition derived from scallop in powder form, which was obtained in Example 4, is 15% larger than the amount of ether type glycerophospholipid contained in the ether type glycerophospholipid composition derived from scallop in powder form, which was obtained in Example 3.

As described above, apparently, the addition of vitamin E enables an increase in the yield of an ether type glycerophospholipid composition in powder form, and addition of an antioxidant such as vitamin E is effective in the preparation of the ether type glycerophospholipid composition in powder form.

### <Test Example 3> Evaluation of Stability

The ether type glycerophospholipid compositions derived from scallop in powder form, which were obtained in the Examples 1 to 4 as well as Comparative examples 1 and 2, were subjected to a stability test in accordance with the following Test method.

### <Test Method>

Approximately 50 mg of the ether type glycerophospholipid composition derived from scallop in powder form thus obtained was dispensed to several 1.5 mL test tubes (sample stock tube T-202, manufactured by BM Equipment Co., Ltd.), and placed in an oven at a temperature of 60° C.

The composition was taken out after the lapse of a predetermined time, and subjected to extraction with methanol.

Subsequently, HPLC analysis was provided on the extract thus obtained under the conditions similar to Example 1.

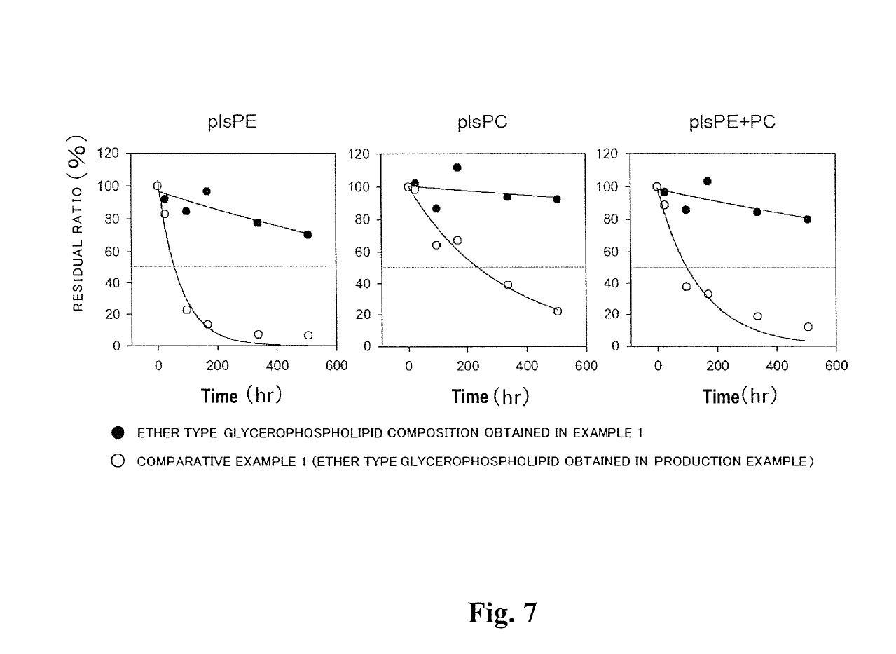

The results are shown in FIGS. 7 to 10.

### <Results>

According to FIG. 7, the ether type glycerophospholipid composition in powder form, which was obtained in Example 1, contains 90 mass % of cyclodextrin relative to 10 mass % of ether type glycerophospholipid. In this case, the residual amount of the ether type glycerophospholipid was approximately 85% of the initial amount after a lapse of 96 hours, and approximately 70% of the initial amount after a lapse of 504 hours.

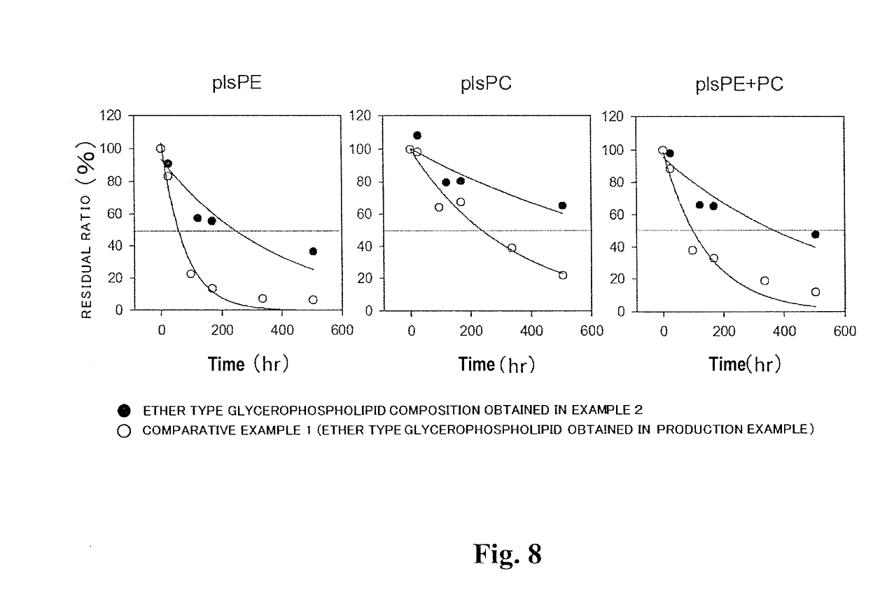

Further, according to FIG. 8, the ether type glycerophospholipid composition in powder form, which was obtained in Example 2, contains 80 mass % of cyclodextrin relative to 20 mass % of ether type glycerophospholipid.

In this case, the residual amount of the ether type glycerophospholipid was approximately 60% of the initial amount after a lapse of 96 hours, and approximately 40% of the initial amount after a lapse of 504 hours.

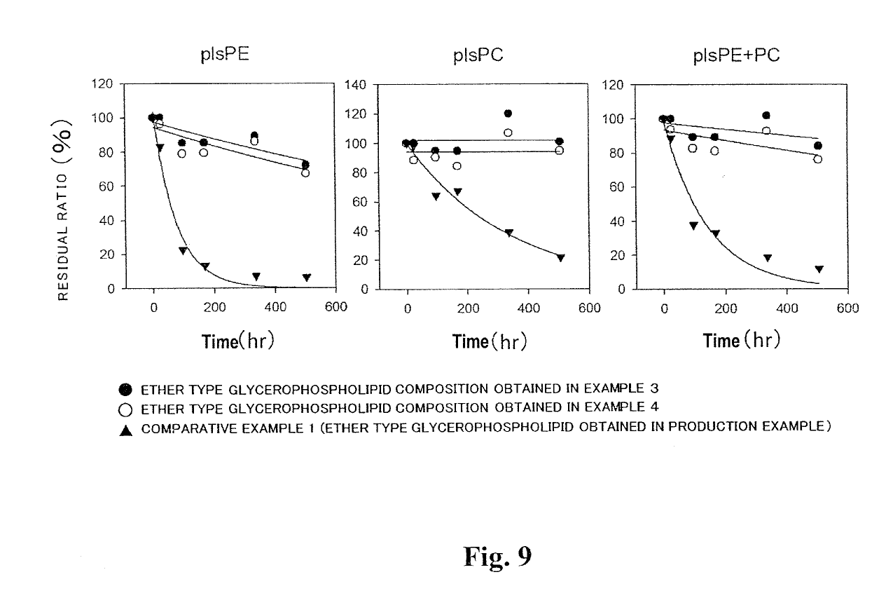

Furthermore, according to FIG. 9, any of the ether type glycerophospholipid compositions in powder form, which were obtained in Examples 3 and 4, contain 91 mass % of cyclodextrin relative to 9% of ether type glycerophospholipid. In these cases, the residual amount of the ether type glycerophospholipid was approximately 85% of the initial amount after a lapse of 96 hours, and approximately 70% of the initial amount after a lapse of 504 hours.

On the other hand, the ether type glycerophospholipid (Comparative example 1) obtained in Production example does not contain cyclodextrin at all.

In this case, according to FIGS. 7 to 10, it is found that the ether type glycerophospholipid degrades with time at a temperature of 60° C.

In particular, the residual amount of ethanolamine type phospholipid decreased to approximately 20% of the initial amount after a lapse of 96 hours, and to as low as 6% of the initial amount after a lapse of 504 hours, and therefore, it is found that ethanolamine type phospholipid has poor thermal stability, compared with the ether type glycerophospholipid compositions according to the present invention.

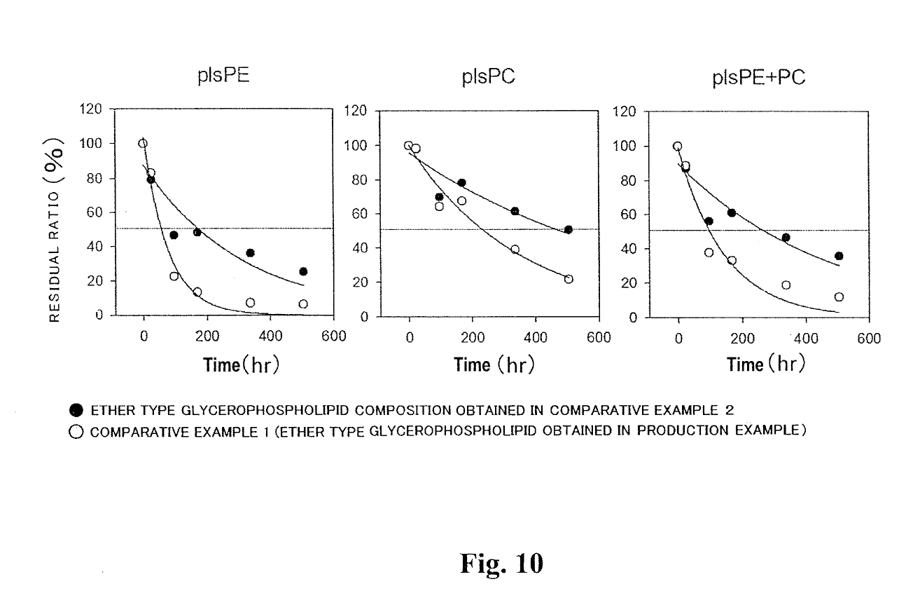

In addition, according to FIG. 10, the ether type glycerophospholipid in powder form, which was obtained in Comparative example 2, contains 75 mass % of cyclodextrin relative to 25 mass % of ether type glycerophospholipid. In this case, the residual amount of ether type glycerophospholipid decreased to approximately 47% of the initial amount after a lapse of 96 hours, and to 25% of the initial amount after a lapse of 504 hours.

As described above, it is found that the composition prepared so as to contain less than 80 mass % of cyclodextrin relative to 20 mass % of ether type glycerophospholipid results in poor thermal stability and poor oxidation stability.

Therefore, apparently, the ether type glycerophospholipid compositions according to the present invention have extremely higher thermal stability and oxidation stability, and this results from containing 20 mass % or less of ether type glycerophospholipid and 80 mass % or more of cyclodextrin.

### INDUSTRIAL APPLICABILITY

According to the present invention, it enable to provide ether type glycerophospholipids, that are effective for improving or preventing Alzheimer's disease and the like, as a composition having high thermal stability and also having oxidation stability and storage stability, especially in powder form. Therefore, the present invention may be applied widely in the medicinal industry.

1-10. (canceled)

11. An ether type glycerophospholipid-containing composition comprising:
20 mass % or less of ether type glycerophospholipid; and
80 mass % or more of cyclodextrin.

12. The ether type glycerophospholipid-containing composition according to claim 11,
wherein the ether type glycerophospholipid is included in the cyclodextrin.

13. The ether type glycerophospholipid-containing composition according to claim 11, being in powder form.

14. The ether type glycerophospholipid-containing composition according to claim 11,
wherein the cyclodextrin is γ-cyclodextrin.

15. The ether type glycerophospholipid-containing composition according to claim 11,
wherein the ether type glycerophospholipid is obtained from a biological material by extraction and purification or is obtained by chemical synthesis.

16. The ether type glycerophospholipid-containing composition according to claim 11, comprising:
less than 20 mass % of ether type glycerophospholipid; and
more than 80 mass % of cyclodextrin.

17. A method for producing an ether type glycerophospholipid-containing composition comprising:
mixing together 20 mass % or less of ether type glycerophospholipid and 80 mass % or more of cyclodextrin in the presence of water and/or ethanol.

18. The method for producing an ether type glycerophospholipid-containing composition according to claim 17,
wherein a mixture obtained by the mixing is subjected to drying.

19. The method for producing an ether type glycerophospholipid-containing composition according to claim 18,
wherein the drying is performed by freeze drying or spray drying.

20. An ether type glycerophospholipid-stabilizing agent
wherein 80 mass % or more of the cyclodextrin is incorporated into 20 mass % or less of ether type glycerophospholipid.

21. The ether type glycerophospholipid-containing composition according to claim 12, being in powder form.

22. The ether type glycerophospholipid-containing composition according to claim 12,
wherein the cyclodextrin is γ-cyclodextrin.

23. The ether type glycerophospholipid-containing composition according to claim 13,
wherein the cyclodextrin is γ-cyclodextrin.

24. The ether type glycerophospholipid-containing composition according to claim 12,
wherein the ether type glycerophospholipid is obtained from a biological material by extraction and purification or is obtained by chemical synthesis.

25. The ether type glycerophospholipid-containing composition according to claim 13,
wherein the ether type glycerophospholipid is obtained from a biological material by extraction and purification or is obtained by chemical synthesis.

26. The ether type glycerophospholipid-containing composition according to claim 14,
wherein the ether type glycerophospholipid is obtained from a biological material by extraction and purification or is obtained by chemical synthesis.
